(19) United States
(12) Patent Application Publication
(10) Pub. No.: US 2013/0072948 A1
(43) Pub. Date: Mar. 21, 2013

(54) SUTURE PASSER DEVICE AND SUTURE NEEDLE

(71) Applicants: Robert G. STATES, III, Morrow, OH (US); Christoph L. GILLUM, Liberty Township, OH (US); David A. PARROTT, Cincinnati, OH (US); Benjamin T. KRUPP, Wyoming, OH (US); Kenneth E. HOGUE, Lebanon, OH (US)

(72) Inventors: Robert G. STATES, III, Morrow, OH (US); Christoph L. GILLUM, Liberty Township, OH (US); David A. PARROTT, Cincinnati, OH (US); Benjamin T. KRUPP, Wyoming, OH (US); Kenneth E. HOGUE, Lebanon, OH (US)

(73) Assignee: COST CONTAINMENT, INC., Albany, NY (US)

(21) Appl. No.: 13/236,215

(22) Filed: Sep. 19, 2011

(51) Int. Cl. A61B 17/04; A61B 17/06

(52) U.S. Cl. 606/145; 606/224

(57) ABSTRACT

A suture passer device includes a shaft, a handle, an end effector, and a suture capturing member. The shaft has a shaft proximal end and a shaft distal end. The handle assembly is coupled to the shaft proximal end. The end effector is coupled to the shaft distal end. The suture capturing member is coaxial with and slideably coupled to the end effector, is coupled to the handle assembly, and is configured to capture a suture within the end effector. A suture needle includes a suture needle body and a suture needle tip, the suture needle body having a first portion and a second portion. The first portion is thicker than the second portion, and the first portion is configured to mate with a needle spacer that enables actuation of a suture needle passer device.

### REFERENCE TO CO-PENDING APPLICATIONS FOR PATENT

The present application for patent is related to co-pending U.S. patent application Ser. No. 12/554,703, titled “Suture Passer Device and Suture Needle”, filed Sep. 4, 2009, assigned to the assignee hereof, and expressly incorporated by reference herein.

### BACKGROUND

1. Field

The present disclosure relates to a suture passer device and, more particularly, to a suture passer device configured to capture a suture.

2. Description of Related Art

A suture passer device is a tool for passing a suture through tissue, such as through a rotator cuff. Several existing suture passer devices provide mechanisms for capturing the suture after the suture passes through the tissue. However, existing passer devices experience fatigue issues. As such, there is a need for a suture passing device with an improved mechanism for capturing a suture and with improved fatigue resistance. Furthermore, there is a need for a corresponding suture needle that works in conjunction with the improved suture passing device.

### SUMMARY

In an aspect of the disclosure, a suture passer device includes a shaft, a handle, an end effector, and a suture capturing member. The shaft has a shaft proximal end and a shaft distal end. The handle assembly is coupled to the shaft proximal end. The end effector is coupled to the shaft distal end. The suture capturing member is coaxial with and slideably coupled to the end effector, is coupled to the handle assembly, and is configured to capture a suture within the end effector.

In an aspect of the disclosure, a suture passer device includes a shaft, a handle assembly, an end effector, and means for capturing a suture. The shaft has a shaft proximal end and a shaft distal end. The handle assembly is coupled to the shaft proximal end. The end effector is coupled to the shaft distal end. The means for capturing a suture is coaxial with and slideably coupled to the end effector and is configured to move responsive to a force applied within the handle assembly.

In an aspect of the disclosure, a suture needle apparatus includes a suture needle, a protrusion, and a circular member. The suture needle includes a suture needle body and a suture needle tip. The suture needle body has a first portion and a second portion, wherein the first portion is thicker than the second portion. The first and/or second portion is configured to mate with a needle spacer that enables actuation of a suture needle passer device. The suture needle body further includes a third portion that is flexible and may be coated with an elastomer. The protrusion extends from the suture needle body substantially transverse to a longitudinal axis of the suture needle body and is configured to mate with a retaining slot configured within a suture passer device. The circular member is attached to an end of the suture needle body and may include the protrusion.

### BRIEF DESCRIPTION OF THE DRAWINGS

FIG. 1 is a side view of an exemplary suture passer device.

FIG. 2 is another side view of the exemplary suture passer device.

FIG. 3 is an exploded view of the exemplary suture passer device.

FIG. 4 is a close-up exploded view of a portion of the exemplary suture passer device.

FIG. 5 is a cutaway view of the exemplary suture passer device.

FIG. 6 is a partial perspective cutaway view of the exemplary suture passer device.

FIG. 7A is a first perspective view demonstrating capture of a suture with the exemplary suture passer device.

FIG. 7B is a close-up of the end effector of the exemplary suture passer device of FIG. 7A.

FIG. 8A is a second perspective view demonstrating capture of a suture with the exemplary suture passer device.

FIG. 8B is a close-up of the end effector of the exemplary suture passer device of FIG. 8A.

FIG. 9A is a third perspective view demonstrating capture of a suture with the exemplary suture passer device.

FIG. 9B is a close-up of the end effector of the exemplary suture passer device of FIG. 9A.

FIG. 10A is a fourth perspective view demonstrating capture of a suture with the exemplary suture passer device.

FIG. 10B is a close-up of the end effector of the exemplary suture passer device of FIG. 10A.

FIG. 11A is a fifth perspective view demonstrating capture of a suture with the exemplary suture passer device.

FIG. 11B is a close-up of the end effector of the exemplary suture passer device of FIG. 11A.

FIG. 12 is a perspective view of the suture needle.

### DETAILED DESCRIPTION

The present invention is described more fully hereinafter with reference to the accompanying drawings, in which various aspects of a suture passer device and a suture needle are shown. This invention, however, may be embodied in many different forms and should not be construed as limited by the various aspects of the suture passer device and the suture needle presented herein. The detailed description of the suture passer device and the suture needle is provided below so that this disclosure will be thorough and complete, and will fully convey the scope of the present invention to those skilled in the art.

The detailed description may include specific details for illustrating various aspects of a suture passer device and a suture needle. However, it will be apparent to those skilled in the art that the invention may be practiced without these specific details. In some instances, well known elements may be omitted to avoid obscuring the inventive concepts presented throughout this disclosure.

Various aspects of a suture passer device and a suture needle may be illustrated with reference to one or more exemplary embodiments. As used herein, the term “exemplary” means “serving as an example, instance, or illustration,” and should not necessarily be construed as preferred or advantageous over other embodiments of the controller disclosed herein. In addition, the term “coupled” means that two elements are connected either directly or indirectly with one or more intervening elements.

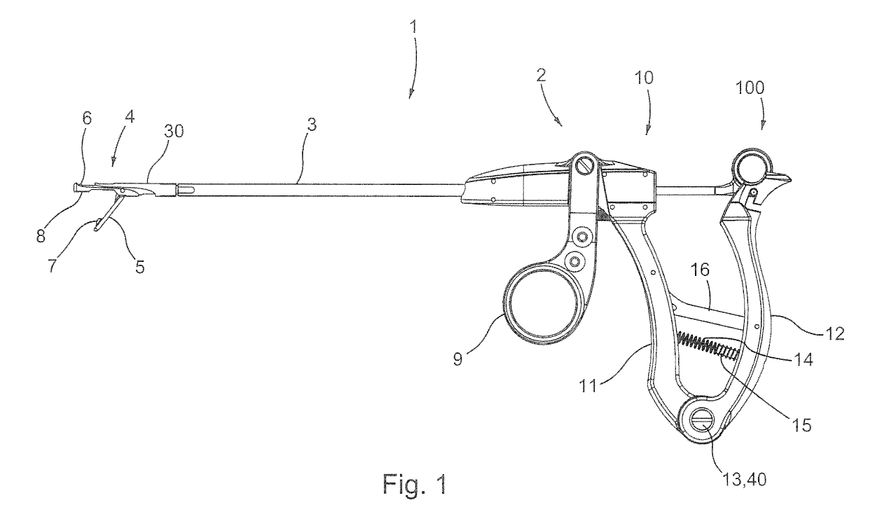

FIG. 1 is a side view of an exemplary suture passer device 1. The suture passer device 1 includes a handle assembly 2, a shaft 3, and an end effector 4. The end effector 4 has a lower jaw 5 and an upper jaw 6. The lower jaw 5 includes ridges 7 between which a suture needle 100 extends and to which a suture needle retracts. The upper jaw 6 may include teeth 8 for gripping tissue/muscle. The shaft 3 may be shaped as a cylinder or a tube. The shaft has a shaft proximal end coupled to the handle assembly 2 and a shaft distal end coupled to the end effector 4. The handle assembly 2 includes a trigger 9 for controlling movement of the lower jaw 5, a front handle assembly 10, and a rear handle 12 connected to the front handle assembly 10. The front handle assembly 10 includes a front handle 11. The rear handle 12 is connected to the front handle assembly 10 at pivot 13. The pivot 13 may be a pin or a bolt 40. A compression spring 14 may be optionally coupled between the front handle assembly 10 and the rear handle 12. The spring 14 may optionally include a guide or bar 15 to keep the spring 14 from buckling. The rear handle 12 may also be connected to the front handle assembly 10 with an optional handle guide 16 for maintaining the alignment of the rear handle 12. The front handle assembly 10 may additionally include a manual cocking lever (not shown) and a manual release button for releasing the cocking lever (not shown). The cocking lever and release button may be implemented in the manner described in related co-pending U.S. patent application Ser. No. 12/554,703. A suture capturing member 30 that is coaxial with and slideably coupled to the end effector 4 and coupled to the handle assembly 10 may be positioned by actuation of the handle assembly 2. The suture passer device 1 is configured to extend a suture needle 100 through the handle assembly 2, the shaft 3, and out the lower jaw 5 of the end effector 4.

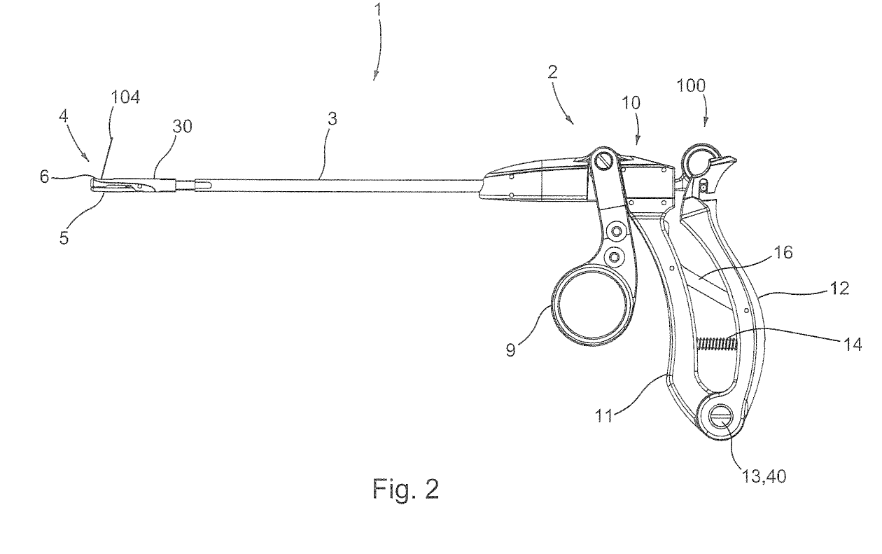

FIG. 2 is another side view of the exemplary suture passer device 1. As shown in FIG. 2, the rear handle 12 is positioned closer to the front handle 11, such that the spring 14 is compressed. In addition, the trigger 9 is pulled back to close the lower jaw 5 against the upper jaw 6. The rear handle 12 extends the suture needle 100 through the handle assembly 2, the shaft 3, and the end effector 4 such that the suture needle 100 extends through the lower and upper jaws 5, 6. Actuation of the suture needle 100 and springs internal to the handle assembly cause the suture capturing member 30 to be positioned to allow a suture and suture needle tip 104 to extend through the lower and upper jaws 5, 6.

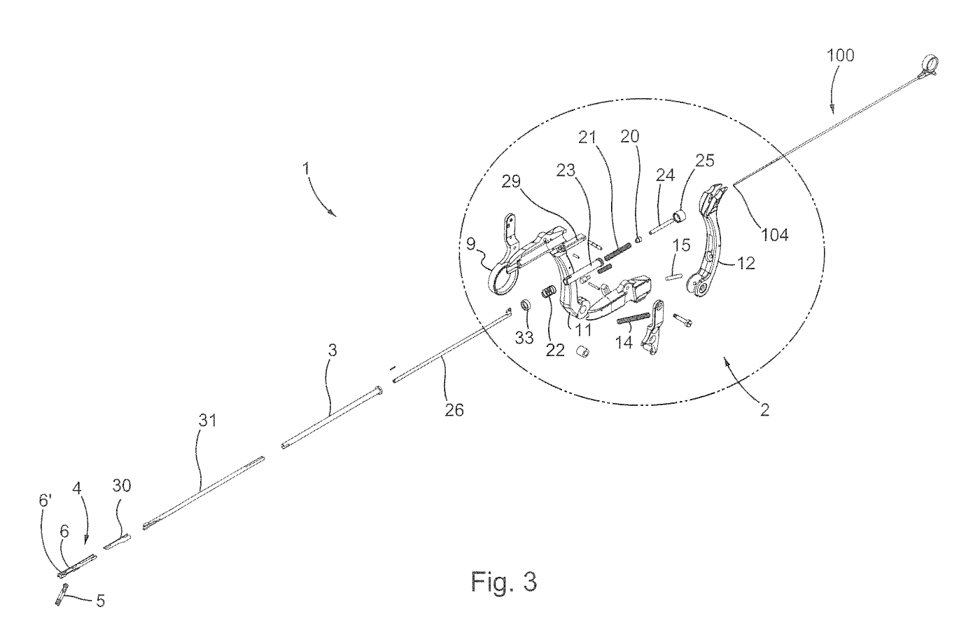

FIG. 3 is an exploded view of the exemplary suture passer device 1. The upper jaw 6 has an opening 6′ through which the suture needle tip 104 can extend. The handle assembly 2 includes a hammer 20, a first spring 21 for biasing the hammer 20 in a first direction, a housing 23 concentric with and enclosing the first spring 21 and coupled with a suture capture tube 31, a second compression spring 22 for biasing the housing 23 along with a coupled backstop 25 in the first direction. The second compression spring 22 may be at least partially concentric with the first spring 21 and may be concentric with the housing 23. The second compression spring 22 is disposed outside of the housing 23. The handle assembly 2 further includes a needle spacer 24 concentric with the suture needle 100 and abutting the hammer 20, and a spring seat 33 coupled to the second compression spring 22. The handle assembly 2 also includes a trigger linkage 29 coupled to the trigger 9. A trigger coupling tube 26, concentric with the shaft 3 may be coupled between the lower jaw 5 and the trigger linkage 29. The trigger coupling tube 26 and the trigger linkage 29 couple the trigger 9 to the lower jaw 5. The suture capturing member 30 may be a partial cylinder coaxial with and slideably coupled to the upper jaw 6 of the end effector 4, and is coupled with the capture tube 31 that extends from the handle assembly 2 and through the shall 3. Accordingly, the suture capturing member 30 is coaxial with and slideably coupled to the upper jaw 6 of the end effector 4 so that during actuation of the suture device the suture capturing member 30 is not subject to a bending stress at any point. The suture capturing member 30 is configured to move/slide independently of the end effector 4 and coaxially with the end effector 4. That is, while the suture capturing member 30 is configured to slide and mate with the upper jaw 6 as the as the lower jaw 5 opens and closes, the suture capturing member 30 is also configured to move longitudinally (e.g. along a length of the upper jaw 6) coaxial with the upper jaw 6, and therefore moves independently of the lower jaw 5 when moving along the upper jaw 6.

It is within the scope of the invention that the suture capturing member 30 may be made of any material having properties suitable to allow repeated actuation and capture of a suture. Because the suture capturing member 30 slides as described herein, the suture capturing member 30 does not experience bending fatigue, and therefore, a wider range of materials may be used as compared to other suture capturing devices where the suture capturing member experiences bending fatigue. Additionally, because the suture capturing member does not experience bending fatigue, the usable life of the suture capturing member is increased as compared to other suture capturing devices where the suture capturing member experiences bending fatigue.

In one configuration, an exemplary suture passer device includes a shaft, a handle assembly, an end effector, and means for capturing a suture. The shaft has a shaft proximal end and a shaft distal end. The handle assembly is coupled to the shaft proximal end. The end effector is coupled to the shaft distal end. The means for capturing a suture is coaxial with and slideably coupled to the end effector and is configured to move responsive to a force applied within the handle assembly. In one configuration, the means for capturing suture is the suture capturing member 30, which may be a partial cylinder that slideably mates with the upper jaw 6 of the end effector and is coupled to a suture capture tube 31 that extends from the handle assembly 2, through the shaft 3.

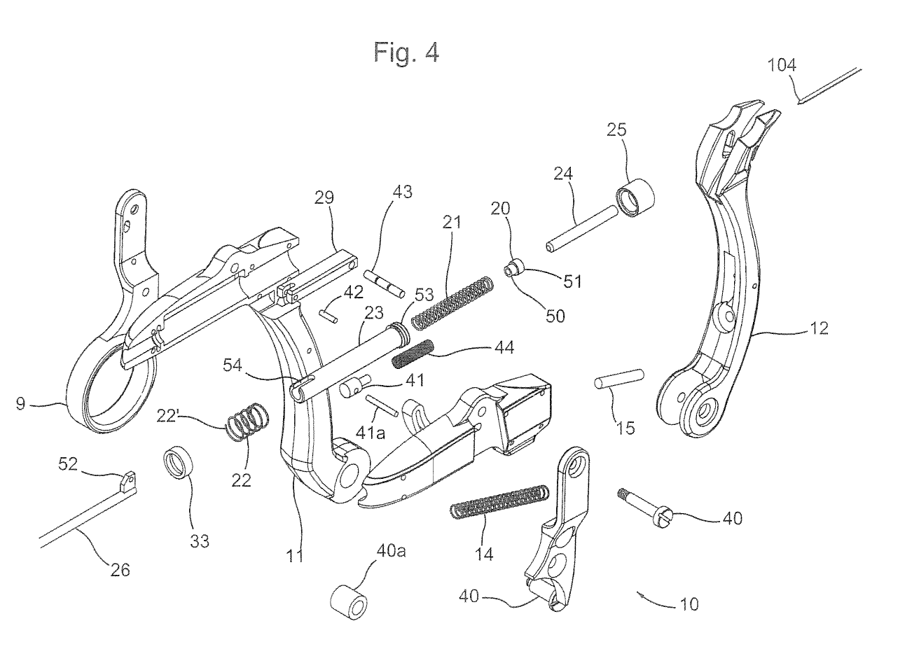

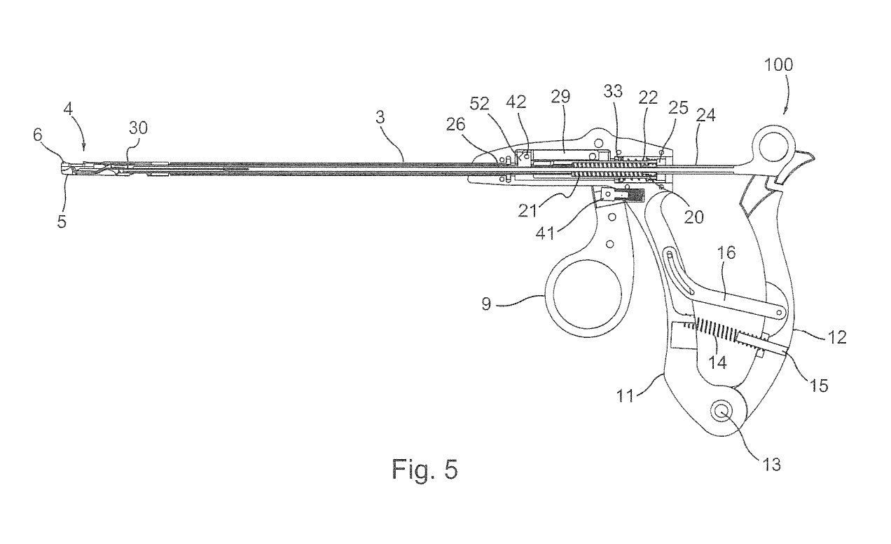

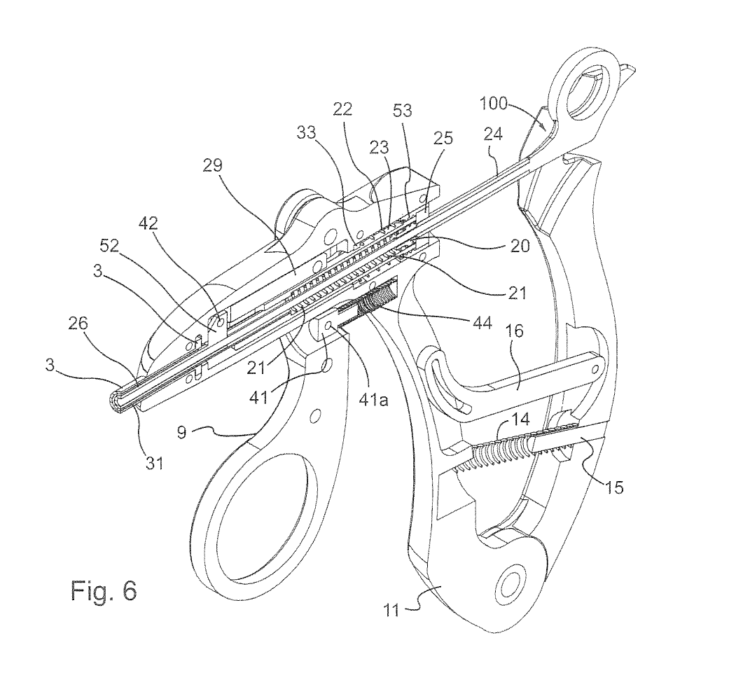

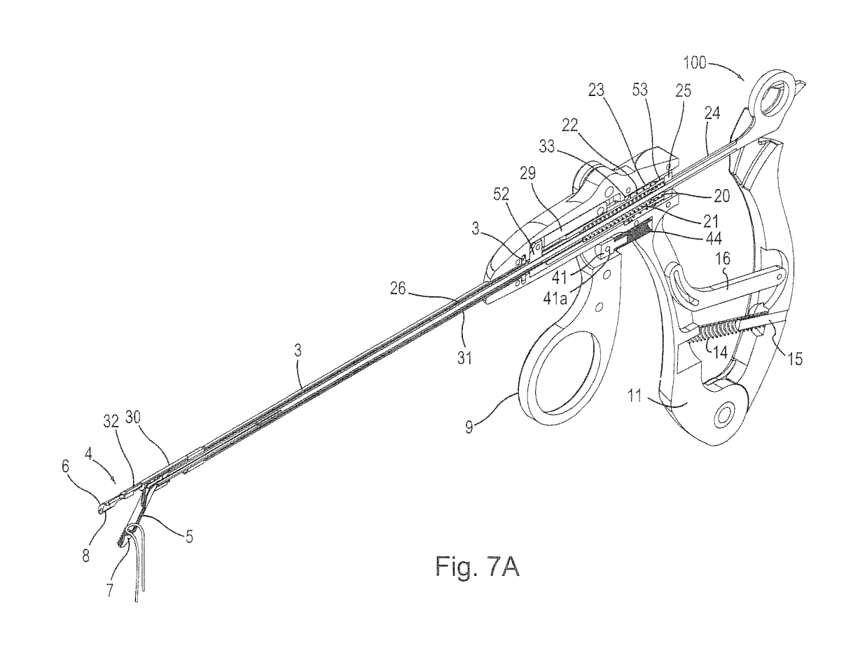

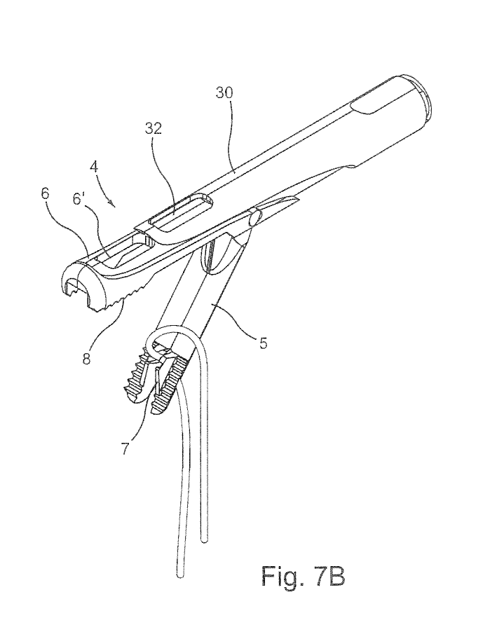

FIG. 4 is a close-up exploded view of a portion of the exemplary suture passer device. FIG. 5 is a cutaway view of the exemplary suture passer device. FIG. 6 is a close-up perspective view of a portion of the exemplary suture passer device. FIGS. 7A through 11B are views of the suture passer device and the suture capturing member 30 throughout the operation states of the suture passer device. The suture capturing member 30 also has an opening 32 through which a suture needle and suture may extend.

As seen in FIGS. 4-6, the front handle assembly 10 is held together with a plurality of bolts 40 and/or press pins and a bushing 40a. The hammer 20 is slideably retained in the spring housing 23 by abutting one side of the first spring 21 on one side of the hammer 20 and abutting the needle spacer 24 on the other side of the hammer 20. The other side of the first spring 21 abuts an end of the suture capture tube 31. The spring housing 23 is slideably retained in the handle assembly by having one end of the housing mating with the backstop 25 while also being coupled with the suture capture tube 31. The backstop 25 mates with one end of the second compression spring 22. The other end of the second compression spring 22 mates with the spring seat 33, which is stationary within the handle assembly. The trigger linkage 29 is coupled to the trigger coupling tube 26 with the pin 42. The capture trigger coupling tube 26 includes flange 52 that rests within a receiving portion 54 of the housing 23. The pin 42 passes through a hole in the flange 52. The pins 43 secure the trigger 9 to the trigger linkage 29 and to the front handle assembly 10. The spring 44 biases the trigger 9 such that the lower jaw 5 is biased in an open position. The spring 44 engages a trigger retainer 41 that is coupled to the trigger 9 via a pin 41a. The components associated with actuating lower jaw 5 make up a lower jaw trigger assembly. The hammer 20 includes a male cylindrical member 50 over which the first spring 21 fits and includes a cylindrical portion 51 which is abutted by the needle spacer 24. The first spring 21 is preloaded (e.g., precompressed) to impart a spring force. The backstop 25 and the spring seat 33 are positioned adjacent opposite ends of the second compression spring 22 around the housing 23. The housing 23 includes a threaded portion 53 allowing the backstop 25 to mate with the housing 23. An edge 22′ of the second spring 22 rests against the spring seat 33. The second spring 22 is also preloaded and is outerly concentric with the first spring 21.

The first spring 21 and the second spring 22 may be configured with different rates (stiffness). The rate of a spring is the change in the force it exerts, divided by the change in deflection of the spring. The inverse of the spring rate is compliance. In one configuration, the first spring 21 has a higher rate (e.g., is stiffer and has a lower compliance) than the second spring 22.

As a force is applied to the hammer 20 by actuation of the handle assembly 10, a force is first exerted on the first spring 21 having the higher stiffness. As the hammer 20 applies force on first spring 21, the first spring 21 in turn applies force on the suture capture tube 31. At this point, before the capture tube 31 moves the capture tube 31 is in a first capture tube position. Likewise, the housing 23 and the backstop 25 are in first positions. Because the suture capture tube 31 may be coupled to housing 23, the force is also transferred to the housing 23. Because the housing 23 may be coupled to the backstop 25 via the threading 53, the force is also transferred to the backstop 25 toward the spring seat 33. However, the backstop 25 may be biased away from the spring seat 33 due to the second spring 22, which has a lower stiffness than the first spring 21. Because the second spring 22 has a lower stiffness than the first spring 21, the initial force applied on the hammer may be entirely transferred to second spring 22. As the second spring 22 is compressed, the backstop 25, along with the housing 23, all of the components inside of the housing 23, the suture needle 100, and the capture tube 31, move axially toward the end effector 4. Because the capture member 30 may be coupled to the capture tube 31, the capture member 30 may slide forward coaxially with the end effector 4 to mate with the upper jaw 6. Thus, the motion of actuating the handle assembly 10 transfers motion across the suture device to allow sliding movement of the capture member 30. Because the second spring 22 has a higher compliance (e.g., lower rate) than the first spring 21, the second spring 22 compresses more easily than the first spring 21, and therefore before the first spring 21 substantially compresses, the housing 23 (and corresponding components) along with the suture needle 100, move axially until the second spring 22 is fully compressed. Once the second spring 22 has been fully compressed, the capture tube 30 has been moved to a second capture tube position and the capture member 30 is in a fully slid position and engaged with the upper jaw 6. Likewise, the housing 23 and the backstop 25 are in second positions.

Upon additional force being applied to the hammer 20, the first spring 21 now compresses, as the housing 23/backstop 25 are no longer able to move axially forward. At this point the hammer 20 is in a first hammer position. Because the second spring 22 is fully compressed there can be no further axial movement of the housing 23 or the suture capture tube 31. However, the hammer 20 and the suture needle 100 are still free to move axially as additional force is provided to overcome the spring force of the first spring 21. As the first spring 21 is further compressed, the suture needle 100 continues to apply force the hammer 20, thereby moving the hammer 20 and the suture needle 100 axially through the suture device until the needle tip 104 extends out from the lower jaw 5, through the opening 32 of the suture capture member 30 and through the opening of the upper jaw 6. Once the first spring 21 is fully compressed, the hammer 20 is in a second hammer position. Thus, generally stated, during the compression of the second spring 22 both the capture member 30 and the suture needle 100 move axially, but after the second spring 22 is fully compressed and the first spring 21 begins to compress, only the suture needle 100 continues to move axially.

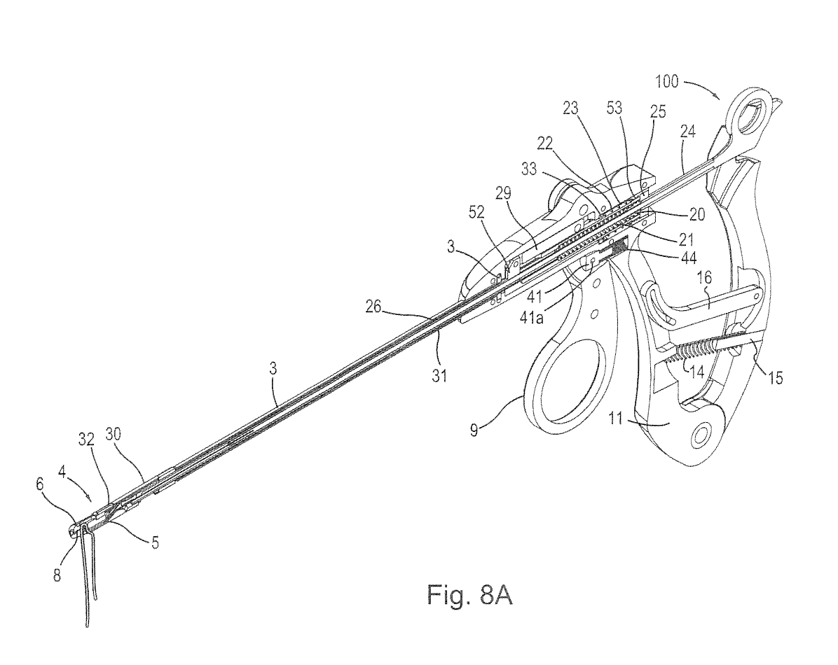

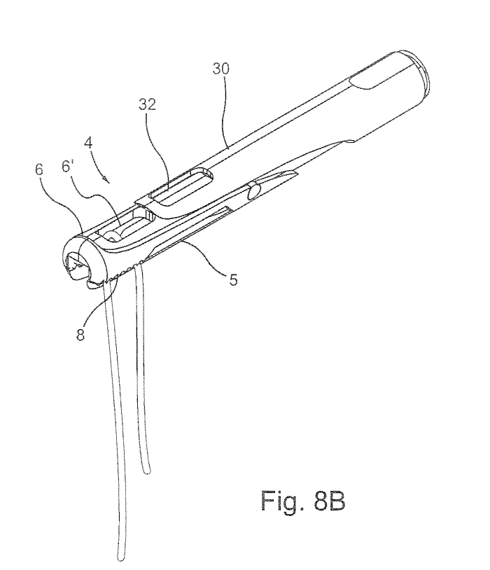

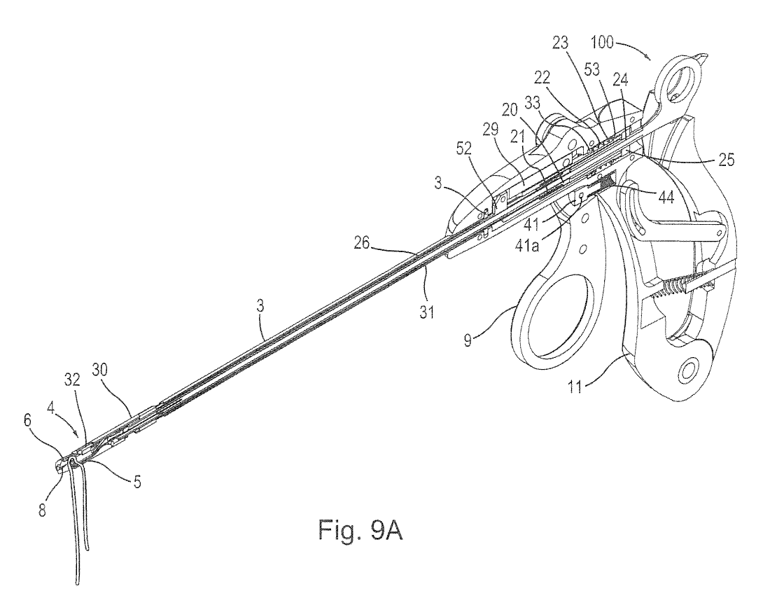

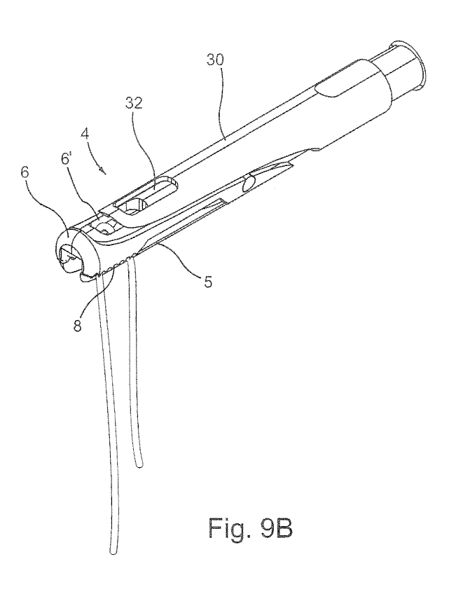

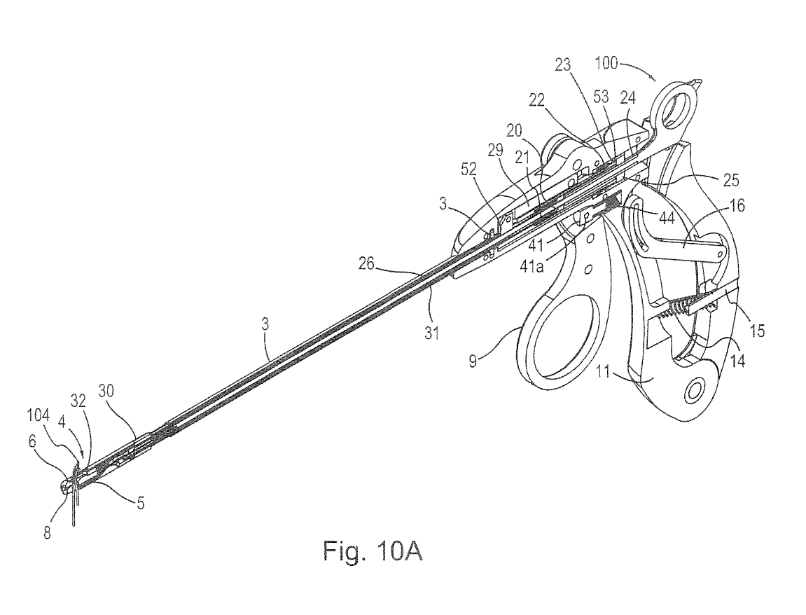

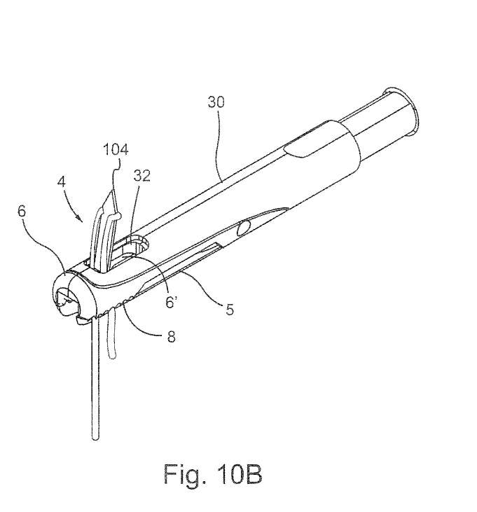

The sequential order of the operation states of the suture device is best seen in FIGS. 7A through 11B. In FIGS. 7A and 7B the suture device has not yet begun operation. The lower jaw 5 is not contacting the upper jaw 6 and the capture member 30 is retracted away from the upper jaw opening 6′. The first spring 21 and the second spring 22 are both fully uncompressed. In FIGS. 8A and 8B, the lower jaw 5 has been made to close against the upper jaw 6 by actuating the trigger 9, but the capture member 30 remains retracted. The first spring 21 and the second spring 22 are both fully uncompressed. In FIGS. 9A and 9B, the capture member 30 is partially slid coaxially with the end effector 4 and is beginning to mate with the upper jaw 6. As illustrated, the opening 32 is partially overlapping the upper jaw opening 6′. This state is achieved by partially actuating the front handle 11, where the second spring 22 has been partially, but not fully compressed. The first spring 21 remains fully uncompressed. In FIGS. 10A and 10B, the capture member 30 has been fully extended coaxially with the end effector 4 and is fully mated with the upper jaw 6 so that the opening 32 coincides with the upper jaw opening 6′. Furthermore, the suture needle 100 has been fully extended such that the suture needle tip 104 has contacted a suture and pushed the suture through both openings 32, 6′. This state is achieved by fully actuating the front handle 11, where the second spring 22 and the first spring 21 have both been fully compressed.

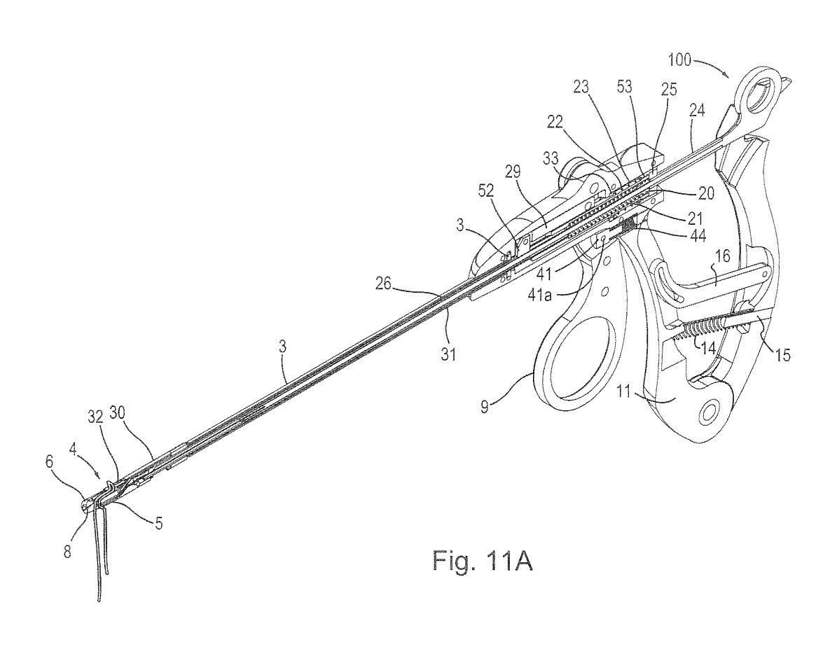

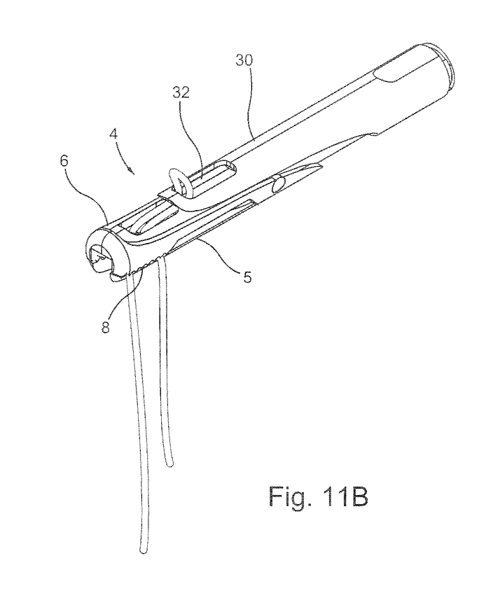

In reverse, as the first spring 21 is decompressed, the suture needle tip retracts back into the lower jaw 5, leaving a suture within the opening 32. As the first spring 21 is further decompressed and the second spring 22 is decompressed, the hammer 20 moves from the compressed position (second position) to uncompressed position (first position) allowing the needle to retract. After the first spring 21 is decompressed, the second spring 22 may begin to decompress, causing the capture member 30 to withdraw coaxially away from the upper jaw 6 thus pinching any suture within the opening 32. This final state of the suture being caught within the capture member 30 is illustrated in FIGS. 11A and 11B.

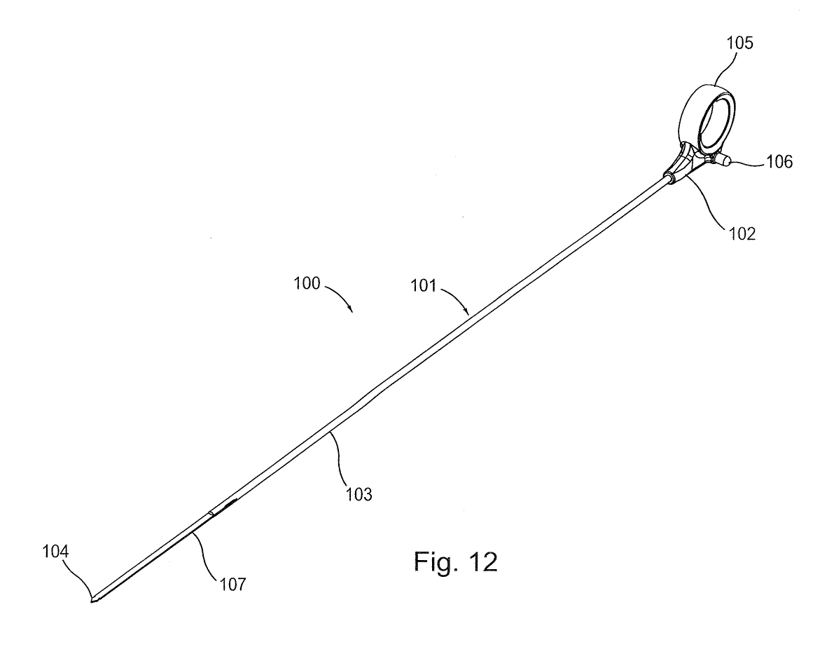

FIG. 12 is a perspective view of the suture needle 100. The suture needle 100 includes a suture needle tip 104 and a suture needle body 101. A protrusion 106 may extend outwardly from the suture needle body 101. The protrusion 106 may be a cylinder shape integral with the suture needle body 101 that extends substantially transverse to the longitudinal axis of the suture needle body 101. A circular member 105 is attached to an end of the suture needle body 101. Although circular member 105 is depicted as a circle, one of ordinary skill in the art would appreciate that shapes other than a circle may be used. The protrusion 106 may be integral with the circular member 105, wherein the circular member 105 with the protrusion 106 is attached to an end of the needle body 101. The protrusions are shaped to allow the needle apparatus to mate with a retaining slot so that the suture needle 100 may be removed, disposed, and replaced when necessary.

The suture needle body 101 may further include a first portion 102 and a second portion 103, wherein the first portion 102 is thicker or has a different geometry than the second portion 103. In particular, as seen in FIG. 12, the first portion 102 may have cylinder shape having a diameter larger than a diameter of the second portion 103. The needle body 101 may further include a third portion 107 have a thin rectangular shape relative to the first and second portions 102, 103. The third portion 107 having a thin rectangular shape allows the needle to move along the lower jaw and up through the upper jaw. The first portion 102, being thicker and cylinder shaped, allows the first portion 102 to interact with the first spring in the manner described above. In particular, the thickness of the first portion 102 is sufficient to abut the needle spacer, which in turn is able to apply force on the hammer. Furthermore, while the above description discloses the needle spacer as a separate element, it is within the scope of the invention that the needle is manufactured such that the needle spacer is integral with the suture needle 100. In other words, the suture needle 100 may be manufactured such that a portion of the second portion 103 is thicker than the rest of the second portion 103 so that that the suture needle 100 may interact with the hammer without an additional needle spacer.

Because the needle bends, as discussed above, the needle apparatus may include a flexible material. Furthermore, the needle may include any material that enhances the useable life cycle or means for preventing the needle tip from breaking off inside a patient. For example, the needle may be coated with a suitable elastomer. When coated with an elastomer, in addition to providing structural integrity, the elastomer may ensure that if a needle tip break occurs, the needle may remain attached to the needle body. Accordingly, the broken needle can be replaced without the needle tip being separated from the needle body inside of the patient. Another manner of preventing the needle tip from breaking off inside of the patient is providing a welded rod along the length of the needle so that if the needle tip breaks it is still attached to the needle body via the welded rod. Other known techniques, such as metallurgical techniques may be implemented to increase the strength of the needle. When one or more of the above techniques are implemented, the disposable life cycle of the suture needle is less than or equal to a disposable life cycle of the suture passer device.

The exemplary suture passing device allows surgeons to control the suture passing process with a single hand while allowing the surgeon to use the other hand to control the arthroscope for visualization, thus facilitating the suture passing process and resulting in a shorter surgery time. A shorter surgery time reduces the amount of time a patient is under anesthesia and reduces the cost of the surgery to both the patient and medical facility.

The various aspects of this disclosure are provided to enable one of ordinary skill in the art to practice the present invention. Modifications to various aspects of a suture passer device and a suture needle presented throughout this disclosure will be readily apparent to those skilled in the art, and the concepts disclosed herein may be extended to other surgical applications. Thus, the claims are not intended to be limited to the various aspects of the suture passer device and the suture needle presented throughout this disclosure, but are to be accorded the full scope consistent with the language of the claims. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A suture passer device, comprising:
a shaft;
a handle assembly coupled to the shaft;
an end effector coupled to the shaft; and
a suture capturing member coaxial with the end effector and slideably coupled to the end effector, coupled to the handle assembly, and configured to capture a suture within the end effector.

2. The suture passer device of claim 1, wherein the suture capturing member comprises a suture capture tube coaxial with the shaft, extending from the end effector to the handle assembly, and configured to move responsive to movement of the handle assembly.

3. The suture passer device of claim 1, wherein the suture capturing member is configured to move independently of the end effector along a longitudinal axis of the shaft.

4. The suture passer device of claim 1, wherein:
the handle assembly is configured to extend and to retract a suture needle apparatus; and
the suture capturing member is configured to slide upon the end effector in response to the suture needle apparatus exerting a force, within the handle assembly, on a capture tube coupled to the suture capturing member.

5. The suture passer device of claim 1, wherein the end effector comprises a lower jaw and an upper jaw with an upper jaw opening.

6. The suture device of claim 5, wherein the handle assembly comprises a lower jaw trigger assembly configured to move the lower jaw toward the upper jaw.

7. The suture device of claim 6, wherein the lower jaw trigger assembly comprises a trigger linkage coupled to a trigger coupling tube, the trigger coupling tube extending within the shaft and coupled to the lower jaw.

8. The suture passer device of claim 5, wherein the handle assembly is configured to extend a suture needle along the lower jaw into the upper jaw opening and to retract the suture needle from the upper jaw opening along the lower jaw.

9. The suture passer device of claim 5, wherein the suture capturing member is configured to capture a suture between the suture capturing member and an inside edge of the upper jaw opening.

10. The suture passer device of claim 7, wherein the suture capturing member has a suture capturing member opening, and the suture capturing member is configured to capture a suture inserted into the upper jaw opening and the suture capturing member opening by pinching the suture between an inside edge of the suture capturing member opening and the inside edge of the upper jaw opening.

11. The suture passer device of claim 1, wherein the handle assembly comprises a hammer coupled to a suture needle apparatus and a housing coupled to the suture capturing member, a first spring biasing the hammer between a first hammer position and a second hammer position, and a second spring biasing the housing in a first housing position and a second housing position.

12. The suture passer device of claim 11, wherein when the housing is in the second housing position, the suture capturing member is positioned to allow a suture to be inserted through the end effector; and when the housing is in the first housing position, the suture capturing member is positioned to capture the inserted suture within the end effector.

13. The suture passer device of claim 11, wherein when the hammer is in the second hammer position, a suture needle is extended out of the end effector; and when the hammer is in the first hammer position, the suture needle is retracted within the end effector.

14. The suture passer device of claim 11, wherein a spring constant of the first spring is greater than a spring constant of the second spring.

15. The suture device of claim 14, wherein at least a portion of the first spring is concentric with at least a portion of the second spring within the handle assembly.

16. The suture passer device of claim 14, wherein:
the end effector comprises a lower jaw and an upper jaw with an upper jaw opening; and
the handle assembly is configured to extend a suture needle apparatus comprising a suture needle along the lower jaw into the upper jaw opening, to retract the suture needle from the upper jaw opening into the lower jaw, and to apply a force via the suture needle apparatus on the first spring; and
the first spring and the second spring are configured to move the housing into the second housing position before the suture needle extends into the upper jaw opening and to delay the housing from moving into the first housing position until the suture needle is retracted from the upper jaw opening.

17. The suture device of claim 16, wherein:
the handle assembly is configured to contact the lower jaw with the upper jaw before the suture needle extends along the lower jaw into the upper jaw opening; and
the lower jaw comprises a ramp configured to direct the suture needle toward the upper jaw opening when the lower jaw is in contact with the upper jaw.

18. The suture device of claim 4, wherein the handle assembly comprises a needle retaining slot and a suture needle comprises a projecting member mateably receivable in the retaining slot.

19. A suture passer device, comprising:
a shaft having a shaft proximal end and a shaft distal end;
a handle assembly coupled to the shaft proximal end;
an end effector coupled to the shaft distal end; and
means for capturing a suture within the end effector and configured to move responsive to a force applied within the handle assembly.

20. The suture passer device of claim 19, wherein the handle assembly comprises a first member coupled to the means for capturing the suture and the means for capturing the suture is configured to move responsive to the first member.

21. The suture passer device of claim 20, wherein:
the handle assembly is configured to extend and to retract a suture needle apparatus; and
the means for capturing a suture is configured to move upon the suture needle apparatus exerting said force on the first member.

22. The suture passer device of claim 20, wherein:
the handle assembly comprises a spring;
the first member is a housing configured to move between a first housing position and a second housing position; and
the housing is biased by the spring between the first housing position and the second housing position.

23. A suture needle apparatus, comprising:
a suture needle comprising a suture needle body and a suture needle tip, the suture needle body having a first portion and a second portion,
wherein the first portion is thicker than the second portion, and
wherein the first portion is configured to mate with a needle spacer that enables actuation of a suture needle passer device.

24. The suture needle apparatus of claim 23, wherein the suture needle body comprises a third portion that is flexible and is configured to bend within a lower jaw of the suture needle passer device and in a direction substantially transverse to a longitudinal axis of the suture need body.

25. The suture needle apparatus of claim 24, wherein at least the third portion comprises an elastomer coating.

26. The suture needle apparatus of claim 23, further comprising a welded rod extending along a length of the suture needle body.

27. The suture needle apparatus of claim 23, further comprising a protrusion configured to mate with a retaining slot configured within the suture passer device that enables the suture needle to capture a suture within an end effector, wherein the protrusion extends substantially transverse to a longitudinal axis of the suture needle body.

28. The suture needle apparatus of claim 25, wherein a disposable life cycle of the suture needle is less than or equal to a disposable life cycle of the suture passer device.
